(19) United States
(12) Patent Application Publication
(10) Pub. No.: US 2018/0325803 A1
(43) Pub. Date: Nov. 15, 2018

(54) USE OF ASCOPHYLLUM NODOSUM EXTRACTS FOR REGULATING EXPRESSION OF GENE GROUPS

(71) Applicant: TCI CO., LTD, TAIPEI (TW)

(72) Inventors: Yung-Hsiang Lin, Taipei (TW); Yu-Ting Lin, Taipei (TW)

(21) Appl. No.: 15/972,708

(22) Filed: May 7, 2018

Related U.S. Application Data
(60) Provisional application No. 62/503,763, filed on May 9, 2017.

(51) Int. Cl. A61K 8/9711; A61Q 17/04

(52) U.S. Cl. CPC A61K 8/9711; A61Q 17/04

(57) ABSTRACT

The present invention provides a method for modulating the expressions of the group of genes, comprising administering to a subject a composition comprising an effective amount of an Ascophyllum nodosum extract, wherein the gene group includes: (i) a Group A gene selected from the group consisting of TGM1, KRT14, FLG, AQP3, GBA, HAS3, and combinations thereof; or (ii) a Group B gene selected from the group consisting of HAS2, MMP2, LOX, and combinations thereof. The Ascophyllum nodosum extract improves skin moisture, skin elasticity, and resistance to ultraviolet radiation.

### CROSS-REFERENCE TO RELATED APPLICATION

This application claims priority of U.S. provisional application No. 62/503,763, filed on May 9, 2017 the content of which is incorporated herein in its entirety by reference.

### BACKGROUND OF THE INVENTION

### 1. Field of the Invention

The present invention relates to a method for modulating the expression of the group of genes, and more particularly to a method for modulating such genes expression by administering an Ascophyllum nodosum extract.

### 2. The Prior Art

The skin is the first line of defense against the damage to the external environment, such as ultraviolet lights, pathogens, and friction, and the moisture loss. From the outside to the inside, the skin consists of the epidermis layer, the dermis layer, which is mainly composed of connective tissue, and the subcutaneous tissue. The epidermis is the outermost layer of the skin and is continuously regenerated. Between the epidermis layer and the dermis layer, there is a continuously dividing cell (such as fibroblasts, keratinocytes, melanocytes) whose activities are very sensitive to UV lights. The dermis contains collagen, elastin, and hyaluronic acid, which impart elasticity and support to the skin. As you age, your skin will experience aging such as wrinkles, fine lines, looseness, depressions, and enlarged pores. The formation of these skin aging phenomena is related to many factors, such as exposure to high levels of ultraviolet light (mainly ultraviolet A) that damages collagen or elastin, and the loss of collagen, elastin, and hyaluronic acid in the dermis with age, all of which reduce the skids fullness and elasticity.

Methods for improving the aforementioned skin aging phenomenon on the market include the use of sunscreens to reduce skin aging caused by ultraviolet lights, direct injection of collagen or hyaluronic acid to the dermis, and oral administration of collagen or hyaluronic acid. However, the chemicals contained in sunscreens may trigger photosensitivity, so that the combination of these chemicals with UV lights has an adverse effect on the skin, such as a rash or more severe sunburn. In addition, the collagen or hyaluronic acid injected into the skin is easily decomposed by enzymes in the body over time, resulting in the need to regularly apply these substances at a high cost. Supplementation of collagen or hyaluronic acid by oral means that these macromolecules are digested into small molecules of amino acids or monosaccharides in the gastrointestinal tract. Although the body can use these amino acids or monosaccharides to synthesize proteins or polysaccharides, it does not necessarily form collagen or hyaluronic acid, so the physical effect of supplementing collagen or hyaluronic acid is limited.

In view of the above, it is necessary to develop a novel composition that is natural and effective in protecting skin from UV damage and delaying skin aging.

### SUMMARY OF THE INVENTION

To solve the foregoing problem, one objective of the present invention is to provide method for modulating the expressions of a group of genes, comprising administering to a subject in need thereof a composition comprising an effective amount of an Ascophyllum nodosum extract, wherein the Ascophyllum nodosum extract is obtained by solvent extraction of a Ascophyllum nodosum, and wherein the gene group includes:
(i) a Group A gene selected from the group consisting of transglutaminase 1 (TGM1), keratin 14 (KRT14), filaggrin (FLG), aquaporin 3 (AQP3), glucocerebrosidase (GBA), hyaluronan synthase 3 (HAS3), and combinations thereof; or
(ii) a Group B gene selected from the group consisting of hyaluronan synthase 2 (HAS2), matrix metalloproteinase 2 (MMP2), lysine oxidase (LOX), and combinations thereof.

In one embodiment of the present invention, the Ascophyllum nodosum extract enhances the gene expression of TGM1, KRT14, FLG, AQP3, GBA, HAS3, HAS2, LOX, and combinations thereof.

In one embodiment of the present invention, the Ascophyllum nodosum extract reduces the gene expression of MMP2.

In one embodiment of the present invention, the solvent is water, alcohol, or a mixture of water and alcohols, and the liquid-to-solid ratio of the solvent to the Ascophyllum nodosum is from 20:1 to 1:1, and the extraction is performed at a temperature from 50 to 100° C.

In one embodiment of the present invention, the Ascophyllum nodosum extract is an Ascophyllum nodosum water extract at a concentration of at least 1 mg/mL.

The Ascophyllum nodosum extract of the present invention obtained by solvent extraction can modulate the gene expression of TGM1, KRT14, FLG, AQP3, GBA, HAS3, HAS2, and LOX to eventually increase the skin moisture and elasticity and to increase the resistance to UV light. The Ascophyllum nodosum extract can be used to prepare a skin care composition such as a food, a drink, a nutritional supplement, or a pharmaceutical composition, and the composition can be in a form selected from the group consisting of powder, granules, liquid, colloid, and cream which is administered to a subject by oral administration or application to the skin.

The present invention is further described in the following examples, in reference to the accompanying drawings. It should be understood that the examples given below do not limit the scope of the invention, and that modifications can be made without departing from the scope of the appended claims.

### BRIEF DESCRIPTION OF THE DRAWINGS

The present invention will be apparent to those skilled in the art from the following detailed description of the preferred embodiments, with reference to the attached drawings, in which:

FIG. 1A shows the relative gene expression levels of TGM1, KRT14, FLG, AQP3, GBA, and HAS3 in human epidermal karatinocyte treated with or without the Ascophyllum nodosum extract.

FIG. 1B shows the relative gene expression levels of MMP2, LOX, and HAS2 in human epidermal karatinocyte treated with or without the Ascophyllum nodosum extract.

FIG. 2 shows the enhancement of the resistance to UVA radiation of the human skin fibroblast treated with the Ascophyllum nodosum extract.

### DETAILED DESCRIPTION OF THE PREFERRED EMBODIMENT

The present invention provides a method using the Ascophyllum nodosum extract for modulating the expressions of a group of genes, wherein the gene group includes: (i) a Group A gene selected from the group consisting of transglutaminase 1 (TGM1), keratin 14 (KRT14), filaggrin (FLG), aquaporin 3 (AQP3), glucocerebrosidase (GBA), hyaluronan synthase 3 (HAS3), and combinations thereof; or (ii) a Group B gene selected from the group consisting of hyaluronan synthase 2 (HAS2), matrix metalloproteinase 2 (MMP2), lysine oxidase (LOX), and combinations thereof. The Ascophyllum nodosum extract is obtained by solvent extraction of an Ascophyllum nodosum, wherein the solvent is water, alcohol, or a mixture of water and alcohols, and the liquid-to-solid ratio of the solvent to the Ascophyllum nodosum is from 20:1 to 1:1, and the extraction is performed at a temperature from 50 to 100° C. The following examples further illustrate the regulation effect of the Ascophyllum nodosum extract on the aforementioned gene group and its effect on improving the UV light resistance of skin fibroblasts to prove the importance of the gene regulation effect on the maintenance of skin health.

### Definition

The data provided in the present invention represent approximated, experimental values that may vary within a range of ±20%, preferably ±10%, and most preferably ±5%.

### Methods and Materials

### Materials

Eagle's minimum essential medium (MEM; catalog number: 61100-053), keratinocyte-SFM (1×) (catalog number: 10724-011), fetal bovine serum (FBS; catalog number: 10437-028), sodium bicarbonate, sodium pyruvate, phosphate buffered saline (PBS solution; catalog number: 14200-075) were purchased from Thermo Fisher Scientific. 3-(4,5-dimethylthiazol-2-yl)-2,5-diphenyltetrazolium bromide (MTT; catalog number: 0793-5G) was purchased from Amersco. Dimethyl sulfoxide (DMSO) was purchased from ECHO Chemical.

### Cell Culture

In one embodiment, human epidermal karatinocyte cells, HPEK-50 cells, purchased from CELLnTEC, and human skin fibroblast, CCD-966SK cells, purchased from Bioresource Collection and Research Center (BCRC 60153) are used for experiments described in the following examples. The HPEK-50 cells were cultured in the SFM at 37° C. under 5% CO₂. The CCD-966SK cells were cultured in the MEM supplemented with 10% FBS, 1.5 g/L sodium bicarbonate, and 1 mM sodium pyruvate at 37° C. under 5% CO₂.

### Gene Expression Analysis

The expression levels of the genes in cells were determined based on quantitative polymerase chain reaction (referred to as qPCR). In brief, ribonucleic acid (RNA) was isolated from cells by using an RNA extraction kit (Geneaid) according to the manufacturer's instructions. The isolated RNA was reverse transcribed to complementary deoxyribonucleic acid (cDNA) at 37° C. by using reverse transcriptase (SuperScript® III, Invitrogen). Thereafter, the cDNA was subjected to PCR amplification by using a qPCR kit (KAPA CYBR FAST qPCR Kit (2×), KAPA Biosystems) and specific primer sets of the target genes and the glyceraldehyde 3-phosphate dehydrogenase (GAPDH) gene as an internal control (Table 1). The qPCR was performed with StepOnePlus™ Real-Time PCR Systems to obtain a melting curve and a cycle threshold (C_{T}) of each gene.

TABLE 1
The sequence of the PCR primer

| | The sequence of the forward primer and the |
|---|---|
| Gene | reverse primer |
| GAPDH | F: CTGGGCTACACTGAGCACC (SEQ ID NO: 1) |
| | R: AAGTGGTCGTTGAGGGCAATG (SEQ ID NO: 2) |
| TGM1 | F: GATCGCATCACCCTTGAGTTAC (SEQ ID NO: 3) |
| | R: GCAGGTTCAGATTCTGCCC (SEQ ID NO: 4) |
| KRT14 | F: TTCTGAACGAGATGCGTGAC (SEQ ID NO: 5) |
| | R: GCAGCTCAATCTCCAGGTTC (SEQ ID NO: 6) |
| FLG | F: GGCAAATCCTGAAGAATCCA (SEQ ID NO: 7) |
| | R: TGCTTTCTGTGCTTGTGTCC (SEQ ID NO: 8) |
| AQP3 | F: GGGGAGATGCTCCACATCC (SEQ ID NO: 9) |
| | R: AAAGGCCAGGTTGATGGTGAG (SEQ ID NO: 10) |
| GBA | F: TCCAGTTGCACAACTTCAGC (SEQ ID NO: 11) |
| | R: TTGTGCTCAGCATAGGCATC (SEQ ID NO: 12) |
| HAS3 | F: CGCAGCAACTTCCATGAGG (SEQ ID NO: 13) |
| | R: AGTCGCACACCTGGATGTAGT (SEQ ID NO: 14) |
| MMP2 | F: GATACCCCTTTGACGGTAAGGA (SEQ ID NO: 15) |
| | R: CCTTCTCCCAAGGTCCATAGC (SEQ ID NO: 16) |
| HAS2 | F: AAGAACAACTTCCACGAAAAGGG (SEQ ID NO: 17) |
| | R: GGCTGGGTCAAGCATAGTGT (SEQ ID NO: 18) |
| LOX | F: CGGCGGAGGAAAACTGTCT (SEQ ID NO: 19) |
| | R: TCGGCTGGGTAAGAAATCTGA (SEQ ID NO: 20) |

Finally, the 2^{-ΔΔCT} method was used to determine the relative expression levels of the target gene. The relative expression levels is defined as the fold change in RNA expression of one target gene in the experimental group relative to the same gene in the control group. This method uses the cycle threshold of the GAPDH gene as the reference threshold for the internal control and calculates the fold change according to the following formula:

ΔCₜ=Ct_{the target gene in experimental or control group}−Ct_{internal control}

ΔΔCₜ=ΔCt_{experimental group}−ΔCt_{control group}

Fold Change=2^{-ΔΔCt Average}

### 3-(4,5-Dimethylthiazol-2-yl)-2,5-diphenyltetrazolium Bromide (MTT) Analysis

Cell viability or proliferation rate was determined by MTT assay. Briefly, 15 μL MTT solution (4 mg/mL MTT in PBS solution) was added to cells in a 96-well plate and reacted at 37° C. for 4 hours. After the reaction solution was removed, 50 μL DMSO was added to the cells and the reaction was shaken for 10 minutes to dissolve the formed formazan crystals. Finally, the absorbance of the cell mixture at 570 nm (OD₅₇₀) was measured by an enzyme-linked immunosorbent assay (ELISA) reader (BioTek).

### Statistical Analysis

The statistically significant difference was determined by the student's t-test of Excel software.

### Example 1

Preparation of the Ascophyllum nodosum Extract

First, Ascophyllum nodosum is washed and processed into an appropriate size by, for example, cutting, grinding, etc., and the treated Ascophyllum nodosum is extracted with a water, alcohol, or alcohol-water mixture as a solvent. The solvent is preferably water, and the liquid-to-solid ratio of the solvent to the Ascophyllum nodosum is from 20:1 to 1:1. The extraction temperature ranges from 50° C. to 100° C., preferably from 80° C. to 95° C. In one embodiment, the time for extraction is from 0.5 to 3 hours.

After the abovementioned extraction, the Ascophyllum nodosum extract is cooled to room temperature and may be further processed by filtered through a 400 mesh filter to remove residual solids. The filtered Ascophyllum nodosum extract may further be concentrated under reduced pressure at 45° C.-70° C. to obtain a concentrated product. In order to produce a solid form of the Ascophyllum nodosum extract, the concentrated product of the Ascophyllum nodosum extract may be subjected to spray drying to remove the solvent, thereby obtaining the powder of the Ascophyllum nodosum extract.

### Example 2

The Ascophyllum nodosum Extract Enhances Expression Levels of Specific Gene Groups in Epidermal Keratinocytes

In order to investigate the regulatory effects of the Ascophyllum nodosum extract on the gene expression in skin cells, in the embodiment, the gene expression changes of the HPEK-50 cells treated with the Ascophyllum nodosum extract were measured by qPCR. HPEK-50 cells were first seeded in 6-well plates at 1.5×10⁵ cells/well and cultured at 37° C. Next, the cells were treated with 2 mg/mL SFM containing 1 mg/ml Ascophyllum nodosum extract (Triplicate Trial), which served as the experimental group. At the same time, the HPEK-50 cells treated with SFM medium without the Ascophyllum nodosum extract was as the control group. After 6 or 24 hours, the cells were collected for qPCR.

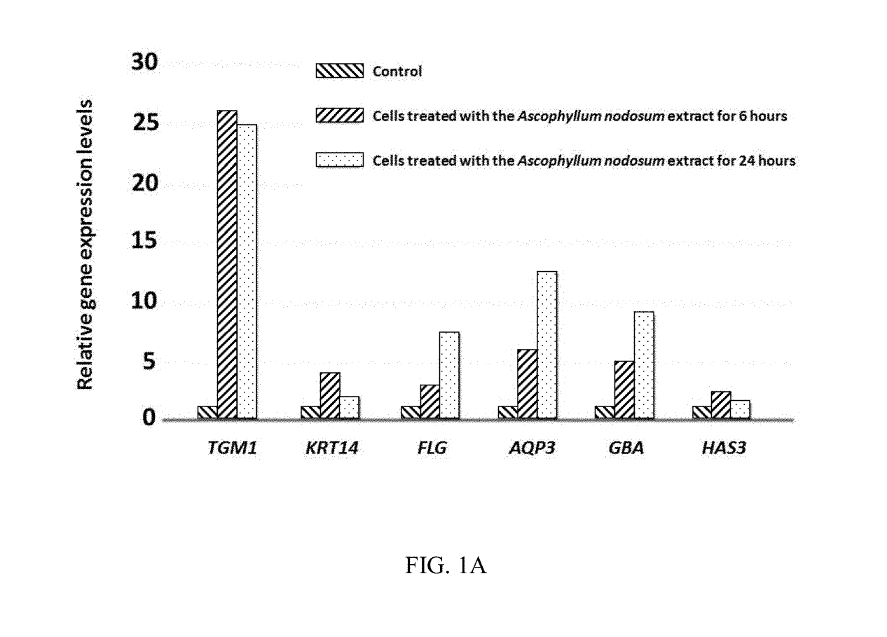

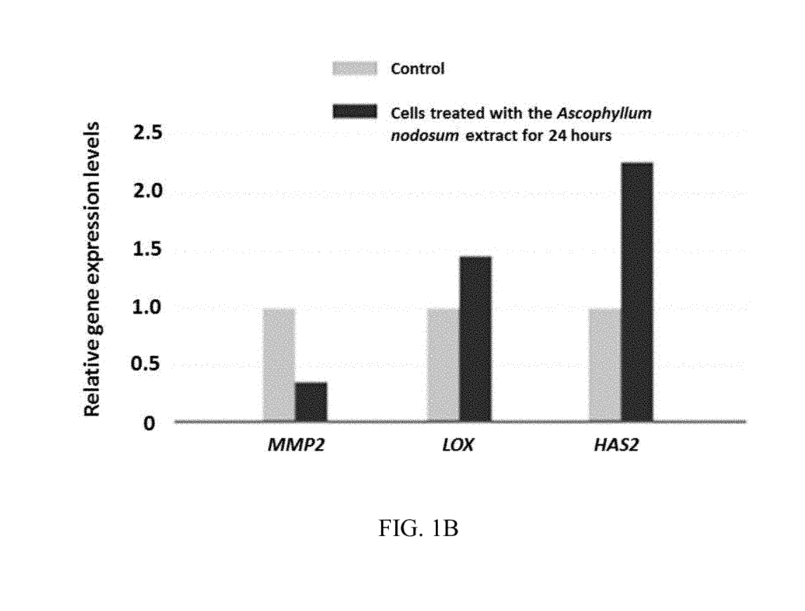

The relative gene expression levels of TGM1, KRT14, FLG, AQP3, GBA, HAS3, HAS2, MMP2, and LOX in the HPEK-50 cells are shown in FIGS. 1A and 1B. As shown in FIG. 1A, compared to the control group, the gene expression of TGM1, KRT14, FLG, AQP3, GBA, and HAS3 significantly increased in HPEK-50 cells treated with 1 mg/ml Ascophyllum nodosum extract for 6 hours or 24 hours. As shown in FIG. 1B, compared to the control group, the gene expression of LOX and HAS3 significantly enhanced and the gene expression of MMP2 significantly inhibited in HPEK-50 cells treated with 1 mg/ml Ascophyllum nodosum extract for 24 hours. Since the up-regulation of TGM1, KRT14, FLG, AQP3, GBA, and HAS3 genes is associated with increased skin barrier and water content, the up-regulation of LOX and HAS3 genes, and the down-regulation of MMP2 gene are associated with improved skin elasticity, the above experimental results indicate that the Ascophyllum nodosum extract is helpful for improving skin moisture and elasticity.

### Example 3

The Ascophyllum nodosum Extract Enhances the Resistance of Skin Fibroblast to UV Light

In order to test whether the Ascophyllum nodosum extract affects the resistance of skin to UV light, the cell viability of the CCD-966SK cells irradiated with UVA were evaluated by a cell survival assay (MTT assay) after treated with the Ascophyllum nodosum extract. Briefly, 5×10³ cells/well of CCD-966SK cells were seeded in 96-well plates with 200 μL MEM and cultured at 37° C. After 24 hours, the cell culture medium was removed, and 200 μL MEM containing 1 mg/mL the Ascophyllum nodosum extract was added to the cells as an experimental group, which was incubated at 37° C. for an additional 24 hours. Afterwards, the cells were irradiated with 12 J/cm² of UVA (wavelength 315-400 nm) in a Vilber for 1 hour, and this radiation dose caused half of the cells to die. At the same time, the CCD-966SK cells irradiated with UVA but treated with the MEM without the Ascophyllum nodosum extract was as the negative control group, and the non-UV-irradiated CCD-966SK cells treated with the MEM without the Ascophyllum nodosum extract was as the mock control group. Finally, perform MTT assay and calculate the cell proliferation rate in each group according to the following formula:

Cell proliferation rate=(OD₅₇₀ of each group/OD₅₇₀ of mock control group)×100%

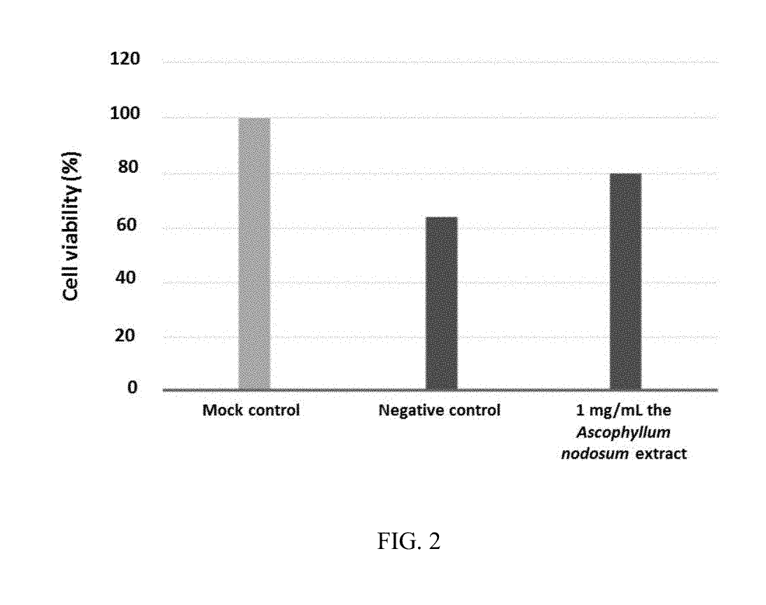

The cell viability of each group of the CCD-966SK cells is shown in FIG. 2. As shown in FIG. 2, the negative control group had significantly decreased cell viability compared to the mock control group, indicating that UVA irradiation caused a large number of skin fibroblast deaths. In contrast, the treatment of the Ascophyllum nodosum extract led to a significant increase in cell viability, suggesting that the Ascophyllum nodosum extract enhances the resistance of the skin to UV light. This result is consistent with the gene expression of the Ascophyllum nodosum extract described in Example 2 that promotes increased skin barrier.

In summary, the Ascophyllum nodosum extract, which is obtained by solvent extraction with water, alcohol, or a mixture of water and alcohol alcohols, can regulate the gene expression levels of TGM1, KRT14, FLG, AQP3, GBA, HAS3, HAS2, MMP2, and LOX, and leads to increased skin moisture and elasticity and the resistance to UV light. The Ascophyllum nodosum extract can be used to prepare a skin care composition such as a food, a drink, a nutritional supplement, and a pharmaceutical composition, and the composition can be in a form selected from the group consisting of powder, granules, liquid, colloid, or cream which is administered to a subject by oral administration or application to the skin.

### SEQUENCE LISTING

### <110> TCI Co., Ltd

### <120> USE OF ASCOPHYLLUM NODOSUM EXTRACTS FOR REGULATING EXPRESSION OF GENE GROUPS

### <130> 107F0272-IE

<160> 20

What is claimed is:

1. A method for modulating the expressions of a group of genes, comprising administering to a subject in need thereof a composition comprising an effective amount of an Ascophyllum nodosum extract, wherein the Ascophyllum nodosum extract is obtained by solvent extraction of a Ascophyllum nodosum, and wherein the gene group includes:
(i) a Group A gene selected from the group consisting of transglutaminase 1 (TGM1), keratin 14 (KRT14), filaggrin (FLG), aquaporin 3 (AQP3), glucocerebrosidase (GBA), hyaluronan synthase 3 (HAS3), and combinations thereof; or
(ii) a Group B gene selected from the group consisting of hyaluronan synthase 2 (HAS2), matrix metalloproteinase 2 (MMP2), lysine oxidase (LOX), and combinations thereof.

2. method according to claim 1, wherein the Ascophyllum nodosum extract enhances the gene expression of TGM1, KRT14, FLG, AQP3, GBA, HAS3, HAS2, LOX, and combinations thereof.

3. The method according to claim 1, wherein the Ascophyllum nodosum extract reduces the gene expression of MMP2.

4. The method according to claim 1, wherein the solvent is water, alcohol, or a mixture of water and alcohols.

5. The method according to claim 1, wherein a liquid-to-solid ratio of the solvent to the Ascophyllum nodosum is from 20:1 to 1:1.

6. The method according to claim 1, wherein the extraction is performed at a temperature from 50 to 100° C.

7. The method according to claim 1, wherein the Ascophyllum nodosum extract is an Ascophyllum nodosum water extract at a concentration of at least 1 mg/mL.

8. The method according to claim 1, wherein the composition is used as a food, a drink, a nutritional supplement, or a pharmaceutical composition.

9. The method according to claim 1, wherein the composition is in a form selected from the group consisting of powder, granules, liquid, colloid, and cream.


SEQUENCE LISTING (20 sequences)

SEQ ID NO: 1 - DNA, 19 bp, Artificial sequence
  PCR primer
ctgggctacactgagcacc

SEQ ID NO: 2 - DNA, 21 bp, Artificial sequence
  PCR primer
aagtggtcgttgagggcaatg

SEQ ID NO: 3 - DNA, 22 bp, Artificial sequence
  PCR primer
gatcgcatcacccttgagttac

SEQ ID NO: 4 - DNA, 19 bp, Artificial sequence
  PCR primer
gcaggttcagattctgccc

SEQ ID NO: 5 - DNA, 20 bp, Artificial sequence
  PCR primer
ttctgaacgagatgcgtgac

SEQ ID NO: 6 - DNA, 20 bp, Artificial sequence
  PCR primer
gcagctcaatctccaggttc

SEQ ID NO: 7 - DNA, 20 bp, Artificial sequence
  PCR primer
ggcaaatcctgaagaatcca

SEQ ID NO: 8 - DNA, 20 bp, Artificial sequence
  PCR primer
tgctttctgtgcttgtgtcc

SEQ ID NO: 9 - DNA, 19 bp, Artificial sequence
  PCR primer
ggggagatgctccacatcc

SEQ ID NO: 10 - DNA, 21 bp, Artificial sequence
  PCR primer
aaaggccaggttgatggtgag

SEQ ID NO: 11 - DNA, 20 bp, Artificial sequence
  PCR primer
tccagttgcacaacttcagc

SEQ ID NO: 12 - DNA, 20 bp, Artificial sequence
  PCR primer
ttgtgctcagcataggcatc

SEQ ID NO: 13 - DNA, 19 bp, Artificial sequence
  PCR primer
cgcagcaacttccatgagg

SEQ ID NO: 14 - DNA, 21 bp, Artificial sequence
  PCR primer
agtcgcacacctggatgtagt

SEQ ID NO: 15 - DNA, 22 bp, Artificial sequence
  PCR primer
gatacccctttgacggtaagga

SEQ ID NO: 16 - DNA, 21 bp, Artificial sequence
  PCR primer
ccttctcccaaggtccatagc

SEQ ID NO: 17 - DNA, 23 bp, Artificial sequence
  PCR primer
aagaacaacttccacgaaaaggg

SEQ ID NO: 18 - DNA, 20 bp, Artificial sequence
  PCR primer
ggctgggtcaagcatagtgt

SEQ ID NO: 19 - DNA, 19 bp, Artificial sequence
  PCR primer
cggcggaggaaaactgtct

SEQ ID NO: 20 - DNA, 21 bp, Artificial sequence
  PCR primer
tcggctgggtaagaaatctga